(19) United States
(12) Patent Application Publication
(10) Pub. No.: US 2025/0195860 A1
(43) Pub. Date: Jun. 19, 2025

(54) CONTINUOUS SEALING TUOHY-BORST ADAPTER

(71) Applicant: Cygnus Medical LLC, Akron, OH (US)

(72) Inventor: Firas Al-Ali, Hudson, OH (US)

(21) Appl. No.: 19/070,749

(22) Filed: Mar. 5, 2025

Related U.S. Application Data
(63) Continuation-in-part of application No. 17/575,780, filed on Jan. 14, 2022.
Application No. 17/575,780 is a continuation-in-part of application No. 16/784,735, filed on Feb. 7, 2020, now Pat. No. 11,376,408.
(60) Provisional application No. 63/187,074, filed on May 11, 2021.

(51) Int. Cl. A61M 39/06

(52) U.S. Cl. CPC A61M 39/0606; A61M 2039/062; A61M 2039/0633; A61M 2205/0216

(57) ABSTRACT

A dynamic radial seal for Touhy-Borst adapters is provided. The seal has an annular base flange with an inwardly directed surface. A plurality of pliable occlusive elements radiate from the inwardly directed surface toward a center and cooperatively form an occlusion with a breaking pressure above typical pressure regimes encountered in vivo in the blood of human subjects.

This application claims the benefit of pending application Ser. No. 17/575,780 filed Jan. 14, 2022 which claims the benefit of application Ser. No. 16/784,735 filed Feb. 7, 2020, now granted U.S. Pat. No. 11,376,408, and application Ser. No. 15/782,664 filed Oct. 12, 2017, now granted U.S. Pat. No. 10,625,067, and provisional patent application No. 62/407,258 filed Oct. 12, 2016 now expired, and provisional patent application 63/187,074 filed May 11, 2021 and now expired. This application further claims the benefit of pending application Ser. No. 17/575,783 filed Jan. 14, 2022, and application Ser. No. 16/784,735 filed Feb. 7, 2020, now granted U.S. Pat. No. 11,376,408, and application Ser. No. 15/782,664 filed on Oct. 12, 2017, now granted U.S. Pat. No. 10,625,067 and provisional patent application No. 62/407,258 filed on Oct. 12, 2016, now expired. application Ser. Nos. 17/575,780; 17/575,783; 16/784,735; 15/782,664; 62/407,258; and 63/187,074 are hereby incorporated by reference in their entireties.

### I. BACKGROUND OF THE INVENTION

### A. Field of Invention

The present invention generally relates to the field of Tuohy-Borst adapters, especially the bleed-back control valves used therein for medical catheterization procedures.

### B. Description of the Related Art

Bleed-back control valves are well-known and have long been in use in surgical intervention and diagnostic procedures involving catheters. They are alternatively known as backflow control valves and hemostasis valves. One common bleed-back control valve is traditionally used in the Tuohy-Borst adapter. Tuohy-Borst adapters are used in catheterization procedures where a catheter is fed into the adapter through distal catheter access port, it travels through the lumen of the adapter, and exits through another port at the proximal end, thus entering the patient. The terms distal and proximal are used herein according to the medial convention relative to the heart, where distal means further from the heart, and proximal means nearer the heart.

Tuohy-Borst adapters include a threaded fitting containing a compressible cylindrical gasket. As the gasket is axially compressed by the fitting, it collapses around the catheter, locking it in place and preventing blood or other fluids from backflowing through the catheter access port. The typical mode of using a Tuohy-Borst adapter is to feed a catheter through the adapter to position it within a patient. Once positioned, the catheter is locked in place.

The Tuohy-Borst adapter is a very common tool in the medical profession even to the extent of being a standard; however, this tool has certain long-standing shortcomings. For instance, bleed-back can only be stopped when the catheter is locked in place. Therefore, as the physician is positioning the catheter within a patient, blood will backflow to some extent. This creates a blood spill, which is undesirable because it increases the risk of exposure to blood-borne pathogens, and because blood loss can have negative consequences for the patient. Generally, the physician will loosen the catheter just enough to allow the catheter to slide. This tends to limit bleed-back, but it does not eliminate it. The bleed-back problem has significant health consequences for both the patient and the medical professionals treating the patient. Nonetheless, the problem has persisted unresolved since the Tuohy-Borst was first introduced in the mid-twentieth century.

What is needed is a bleed-back control valve that slideably engages a catheter while simultaneously blocking bleed-back. Some embodiments of the present invention may provide one or more benefits or advantages over the prior art.

### II. Summary of the Invention

Embodiments of the invention include a dynamic radial Touhy-Borst seal, comprising: an annular base flange having an annular planar surface oriented radially inward; and a plurality of inwardly directed pliable occlusive elements together forming an occlusion with a breaking pressure above physiological blood pressure.

Embodiments further include a dynamic radial Tuohy-Borst seal, comprising an annular base flange having an annular planar surface oriented radially inward; and a plurality of pliable cilia radiating inward from the annular planar surface, and arranged in a regular pattern relative to each other on the annular planar surface, wherein the plurality of pliable cilia form an occlusion in a liquid flow path of the annular base flange with a breaking pressure above physiological blood pressure.

Other benefits and advantages will become apparent to those skilled in the art to which it pertains upon reading and understanding of the following detailed specification.

### III. BRIEF DESCRIPTION OF THE DRAWINGS

The invention may take physical form in certain parts and arrangement of parts, embodiments of which will be described in detail in this specification and illustrated in the accompanying drawings which form a part hereof, wherein like reference numerals indicate like structure, and wherein:

FIG. 1A is a cross sectional view of a valve according to an embodiment of the invention;

FIG. 1B is a cross sectional view of the valve of FIG. 1A receiving a catheter;

FIG. 2 is an exploded view of the valve of FIGS. 1A and 1B;

FIG. 3A is a top view of a seal according to an embodiment of the invention;

FIG. 3B is a side view of the seal of FIG. A;

FIG. 3C is a bottom view of the seal of FIG. A;

FIG. 3D is a second side view of the seal of FIG. A;

FIG. 3E is an elevation view of the seal of FIG. A;

FIG. 4 is a cross-sectional view of a second Tuohy-Borst embodiment containing a second radial seal embodiment;

FIG. 5 is a view of the second radial seal embodiment of FIG. 4; and

FIG. 6 is a view of the second radial seal embodiment in an unrolled configuration.

### IV. DETAILED DESCRIPTION OF THE INVENTION

As used herein the terms “embodiment”, “embodiments”, “some embodiments”, “other embodiments” and so on are not exclusive of one another. Except where there is an explicit statement to the contrary, all descriptions of the features and elements of the various embodiments disclosed herein may be combined in all operable combinations thereof.

Language used herein to describe process steps may include words such as “then” which suggest an order of operations; however, one skilled in the art will appreciate that the use of such terms is often a matter of convenience and does not necessarily limit the process being described to a particular order of steps.

Conjunctions and combinations of conjunctions (e.g. “and/or”) are used herein when reciting elements and characteristics of embodiments; however, unless specifically stated to the contrary or required by context, “and”, “or” and “and/or” are interchangeable and do not necessarily require every element of a list or only one element of a list to the exclusion of others.

The terms distal and proximal are used herein to indicate the relative position or orientation of parts of an embodiment in an assembled state, and/or while in use. Their meaning will be clear in context to the ordinarily skilled artisan, but in general they refer to the direction of travel of a catheter as it is inserted into an embodiment.

The term blood pressure and physiological blood pressure is meant to indicate typical pressure regimes encountered in human subjects.

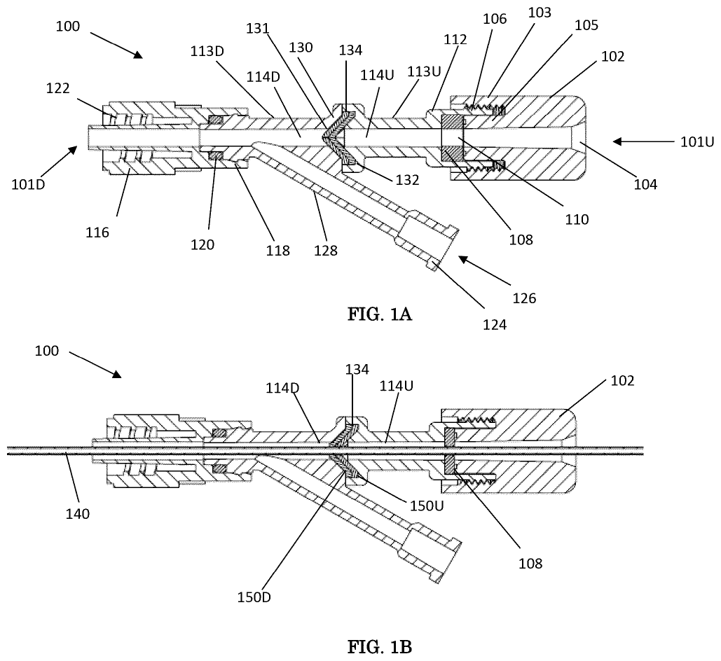

Referring now to the drawings wherein the showings are for purposes of illustrating embodiments of the invention only and not for purposes of limiting the same, FIG. 1A is a cross-sectional view of an embodiment 100 of the invention comprising a fully assembled Tuohy-Borst adapter with an improved dynamic radial seal. The distal end 101U is shown to the right, and the proximal end 101D is on the left. The distal end 101U includes a chamfered, or beveled, catheter access port 104 formed in a compression nut 102. The nut 102 has female threads 103 proximal of the access port 104. The nut also includes a plunger 105 that functions to axially compress a cylindrical gasket 108 as the nut 102 is tightened onto a male thread 106 of the valve body 113U. The cylindrical seal is compressed between the plunger 105 and a seat 112 formed in the valve body 113U. A central through-hole 110 of the cylindrical seal 108 is aligned coaxially with the lumen 114U, 114D which is defined by an inner luminal wall of the valve body 113U, 113D. Thus, a catheter may enter at the distal end, through the access port 104, pass through the through-hole 110, enter the lumen 114U, 114D and exit the valve body at the proximal end 101D of the Tuohy-Borst. The central through-hole 110 is sized to slideably receive the catheter in an uncompressed state. In this context, the term slideably receive means that the catheter is free to travel through the central through hole 110 regardless of whether the catheter actually makes sliding contact with the sides of the through-hole 110.

The valve body is divided into two halves, namely a distal half 113U and a proximal half 113D. The reason for dividing the valve body in this way is to provide structure for easily installing a dynamic radial seal, which in this embodiment is a double conical seal 134, into a mounting groove 132 formed by the two halves. While the present embodiment is divided into two halves, the skilled artisan will readily understand that any of a wide variety of known structures for retaining a seal would also be appropriate as a matter of design choice. Such variations are well within the scope of the present invention as described and claimed herein. FIG. 1A shows a double conical seal 134 held in a mounted relation by the mounting groove 132.

In the embodiment of FIG. 1A, the groove 132 holding the double conical seal has a complex frustoconical-shaped wall 130 formed in the distal and proximal halves of the valve body 113U, 113D. In addition to holding the double conical seal 134 in place, this shape also tends to support a portion of the seal 134 while allowing the apex of the seal to protrude through an orifice 131 and into the proximal lumen 114D. This arrangement may be advantageous by, for instance and without limitation, limiting the amount of flexure that the seal experiences during insertion of a catheter and/or providing improved sealing around a catheter by stiffening the seal and thereby increasing sealing force.

With continued reference to FIG. 1A, the proximal valve body 113D terminates in a rotatable collar fitting comprising an annular ridge and groove connection 118 to a standard Luer Lock fitting 116 threaded 122 to fixedly cooperate with cannulas. By fixedly cooperate, it is contemplated that the threads of the Luer Lock fitting may receive a cannula having complementary structure in a fastened and thus fixed relation relative to the Luer Lock fitting. The fitting 116 is sealed with an O-ring 120 to prevent leakage of fluids from the lumen 114D, 114U. Some embodiments, including the one shown in FIG. 1A, may include a sidearm flush 128 with a port 124 co-operable with standard fluid delivery devices such as syringes. The lumen 126 of the sidearm flush is shown in fluid communication with the proximal lumen 114D of the proximal valve body 113D.

In contrast to FIG. 1A, FIG. 1B illustrates the same embodiment 100 receiving a catheter 140. The catheter is shown locked in place by the cylindrical seal 108 which has been compressed by tightening the nut 102. Accordingly, the seal 108 has collapsed around the catheter 140 and thus locks it in place through friction. FIG. 1B also illustrates the distal conical gasket 150U and the proximal conical gasket 150D of the double conical seal 134 opening at their apexes to receive the catheter 140. The gaskets 150U, 150D dynamically radially seal against the catheter 140 as it is inserted into the embodiment 100 and fed into a patient. The gaskets 150U, 150D then statically maintain the seal when the catheter 140 is locked in place, as shown here.

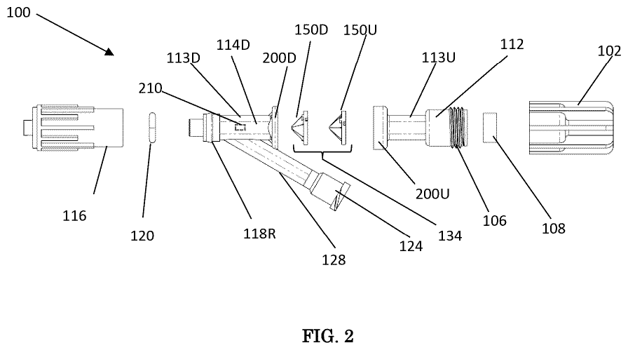

FIG. 2 is an exploded view of the embodiment shown in FIGS. 1A and 1B. The valve body is shown divided into its distal 113U and proximal 113D halves. The distal valve body 113U includes a seat 112 receiving a cylindrical seal 108. A nut 102 is threaded onto the male thread 106 of the distal valve body 103U, which compresses the cylindrical seal 108 with a plunger 105 (see FIG. 1A). Interposed between the two halves of the valve body 113U, 113D are two conical gaskets. One is a distal conical gasket 150U and the other is a proximal conical gasket 150D. The base of the distal gasket 150U fits into a seat 200 at one end of the distal valve body 113U. The two gaskets 150U, 150D stack one within the other, and their angular orientation relative to each other is set by registering structures, as will be described in more detail below.

The conical gaskets 150U, 150D are mounted between a distal flange 200U and a proximal flange 200D. The distal and proximal flanges 200U, 200D include the frustoconical wall 130 and groove 132 which are not visible in this figure, but which can be seen in FIG. 1A. The proximal end of the proximal valve body 113D terminates in a ridge 118R of the ridge and groove connection 118 shown in FIG. 1A. The ridge 118R receives the Luer Lock collar fitting 116 in a rotatable relation sealed with an O-ring 120.

A pressure transducer 210 is shown mounted within the lumen 114D of the proximal valve body 113D. The transducer advantageously has a thin profile which allows it to be in the lumen without occluding or obstructing. Thus, the transducer cooperates with a catheter 140 in that it does not obstruct its path. Accordingly, the transducer is capable of obtaining real time measurements of body fluid pressures while carrying out a procedure without the need for additional fluidics, and without the need to pause the procedure to measure pressure. Suitable pressure transducers are well known in the art and may be selected as a matter of design choice. Optionally, the transducer 210 may include or communicate with electronic components for wirelessly broadcasting telemetry data. The skilled artisan will appreciate that the placement of the transducer 210 is advantageously within the proximal lumen 114D because the distal lumen 114U is isolated by the double conical seal 134.

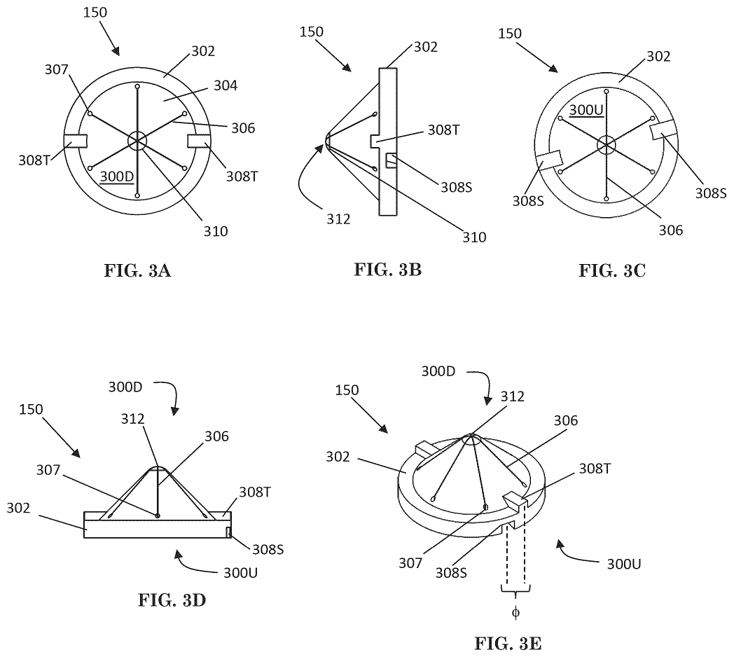

FIGS. 3A through 3E illustrate the same conical gasket 150 in various orientations. The embodiment illustrated in FIGS. 1-2 illustrates a double conical seal 134 which is made of a stacked pair of this conical gasket 150 which, in FIGS. 1-2, are labeled distal 150U and proximal 150D. Their unique reference numbers 150U and 150D are intended only to indicate their position in the assembled device. In this embodiment, the distal and proximal conical gaskets are structurally identical to each other and to the gasket illustrated in FIGS. 3A-3E. The skilled artisan will readily appreciate that being identical is not a requirement, but that certain manufacturing efficiencies are gained by having two of a single part rather than two different parts. Alternatively, the distal gasket and the proximal gasket may be different in that each may limit its registering structures to those configured to engage the other.

With collective reference to FIGS. 3A-3E a conical gasket 150 is shown that has an annular base flange 302. The base flange 302 cooperates with the groove defined in the distal and proximal flanges 200U, 200D of FIG. 2. The distal surface of a conical wall 300U and the proximal surface of the same wall 300D are shown divided into six equal semi-conical flaps 304 through the apex 312. The edges of each semi-conical flap 304 abut the edges of its nearest neighbors to form seams 306. As used in this context, the term seam is intended only to denote area where flap edges abut one another, and it is not intended to imply that the edges are joined. To the contrary, the edges are not joined, and thus the flaps 304 can spread apart in response to an impinging catheter to form an opening at the apex 312 where the catheter may pass through.

The circle 310 is not a structural element of the conical gasket 150. Rather, it is intended to indicate the region where the conical wall 300U, 300D begins to curve to form the blunted apex 312 shown most clearly in FIGS. 3B, 3D, and 3E.

Each seam 306 terminates in a circular through-hole 307 near the base flange 302. This structure is optional, but may be advantageous in preventing tearing of the gasket at the seam terminuses. The gasket 150 has a pair of register tabs 308T located on the proximal surface 180 degrees apart from each other. Similarly, the illustrated embodiment includes a pair of register sockets 308S located on the distal surface 180 degrees apart. Thus, a pair of the gasket 150 may be stacked such that the tabs 308T of one cooperatively fit into the sockets 308S of the other. Register tabs 308T and register sockets 308S are referred to herein according to their genus as register structures, or registering structures. Thus, the angular orientation of the gaskets relative to each other may be fixed.

The skilled artisan will readily appreciate that the number and distribution of register tabs and register sockets may vary. Embodiments may have only one register tab 308T and/or one register socket 308S provided that they are positioned to cooperate with the tabs and sockets of other gaskets 150. Alternatively, embodiments may have a plurality of tabs and sockets, and they may be disposed on either the distal or proximal surface, or even on both surfaces.

With further regard to FIGS. 3A-3E the register tabs 308T and register sockets 308S are shown off-set from each other by an angle ¢. The precise magnitude of the off-set is not critical; however, it should be sufficient to cause the seams 306 of two stacked gaskets 150 to be sufficiently off-set from each other to allow the semi-conical flaps 304 to elastically spread under normal operating conditions, where the embodiment is sealably receiving a catheter, without causing bleed-back of body fluids into the distal lumen 114U. In other words, the gap formed by one gasket's spreading flaps is filled by the flap of the adjacent gasket. Suitable magnitudes will depend in part on the number of semi-conical flaps 304, which may be more or fewer than the illustrated number without departing from the scope of the invention. The skilled artisan will appreciate that a greater number of flaps 304 requires more seams 306 which requires smaller angular off-sets. Suitable magnitudes for $ according to the illustrated embodiment include any angle from 1° to 59°. Other ranges within the scope of the invention include 1° to 5°, 5° to 10°, 10° to 15°, 15° to 20°, 20° to 25°, 25° to 30°, 30° to 35°, 35° to 40°, 40° to 45°, 45° to 50°, 50° to 55°, 55° to 59°, or any combination thereof.

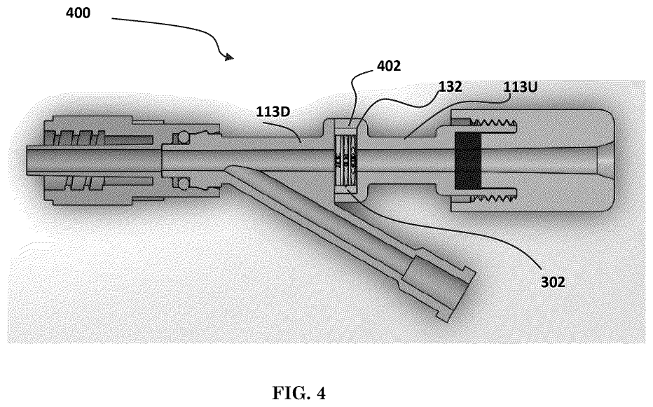

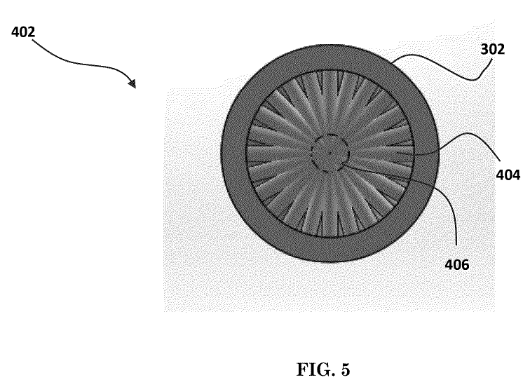

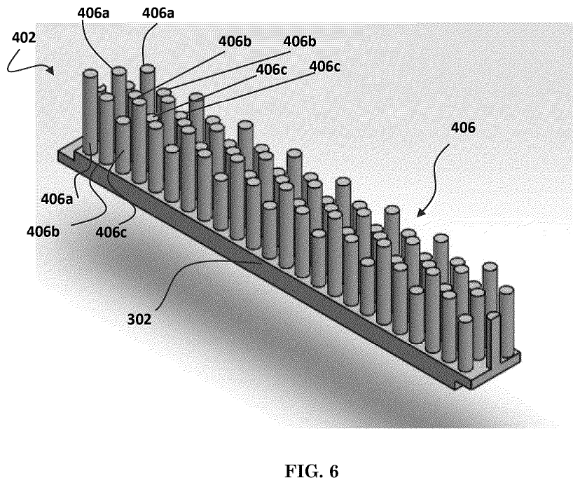

FIG. 4 shows a second Tuohy-Borst adapter embodiment 400. The difference between this and embodiment 100 is radial seal 402. Radial seal 402 operates similarly to that of the double conical seal discussed above; however, the flaps are replaced by cilia, as shown more clearly in FIGS. 5 and 6. FIG. 5 shows that the seal 402 includes an annular base flange 302 adapted to engage a mounting groove 132 formed by the distal half 113U and a proximal half 113D of the Tuohy-Borst embodiment 400. The annular base flange has an annular planar surface oriented radially inward. In other words, as shown in FIGS. 5 and 6, the base flange is the equivalent of a flat surface rolled into an annulus. Other embodiments need not have a planar annular surface. Rather, such surface may be replaced by a secondary curvature perpendicular to the curve of the annulus. Such embodiment may take on a toroidal form, or a complex form having characteristics of a torus. In the illustrated embodiment, the annular planar surface is oriented radially inward in the sense that a line drawn normal to the surface would pass through a center of the seal 402 where the seal is essentially circular. This description of the orientation of the surface is not meant to limit the invention to perfectly circular embodiments or perfectly regular surfaces. The person skilled in the art will readily understand that embodiments are operational within tolerances as the skilled artisan can determine as a matter of design choice.

The seal 402 further comprises a plurality of inward-facing cilia 404 which function similar to the semi-conical flaps 304 of other embodiments herein. Unlike the semi-conical flaps 304, the inward facing cilia 404 are directed more or less radially inward rather than at an oblique angle. Therefore, as shown in the side view of FIG. 4, the seal 402 is a flat annulus rather than conical in shape when viewed side-on. FIG. 5 shows that the cilia converge in a central location 406 within the annulus 302, which occludes the fluid pathway that would otherwise be present inside the annular base flange. Furthermore, the breaking pressure of the occlusion is above physiological blood pressure. As used here, the term breaking pressure means the pressure where a seal is overcome. The person having ordinary skill in the art will understand that breaking pressure is typically represented as a range about an average e.g., 300 mmHg+/−10%. Additionally, the breaking pressure is that which is measured in blood. Breaking pressure varies with the liquid that the seal is challenged to resist. Due to effects of viscosity, surface tension, polarity and other physical factors.

When a microcatheter is inserted through the seal embodiment 402, the seal impinges radially upon the catheter thus forming a seal. Moreover, the seal is effective even when the microcatheter is moving axially through the Tuohy-Borst, relative to the seal 402. Axial motion is enabled by the small radial sealing force, which produces relatively little friction, leaving the catheter free to move while maintaining a dynamic seal. The sealing force is limited in part by the Youngs Modulus of the material composing the cilia, which is typically a silicone, although the invention is not limited to silicones. The cilia are pliable structures that bend easily under shearing forces like that of an axially impinging catheter. The small elastic constant tending to resist flexure is enough to hold back blood under physiological conditions. Meaning, though the elastic constant is small, a patient's blood pressure is smaller. Therefore, the stiffness of the pliable cilia is enough to resist blood flow under both static and dynamic sealing conditions.

Turning to FIG. 6, a seal embodiment 402 is shown unrolled. The base flange 302 is severed in this view, allowing the cilia 406 to all face in the same direction. According to FIG. 6, the cilia are different lengths. The function of variable length is to avoid overcrowding the cilia, which would otherwise cause them to bunch up, creating areas of excess material that could result in binding up the catheter, or tearing or coring of the pliable cilia by the catheter. Such excess material can be problematic. Therefore, the cilia lengths are staggered so that each longest cilium is surrounded by shorter cilia. More specifically, as shown in this particular embodiment, starting at the far left, a row of cilia starts with a longest cilium 406a, followed by an adjacent medium cilium 406b, and then a short cilium 406c. This pattern repeats through the entire row of cilia. As used here, the term medium cilium means a length intermediate between the longest and the shortest cilia. It is not intended to limit medium cilia to a mathematical median or average or any other specific length. Further, length is meant to indicate its full length from the base of a cilium at the annular planar surface to its tip.

An adjacent row begins at the far left of FIG. 6 with a short cilium 406c which is out of view, and the pattern repeats as in the first row. Therefore the rows are offset by one cilium. The third row starts with a medium cilium 406b, which is also out of view, and the pattern repeats, thus incrementing the offset so that the third row is offset by two cilia from the first row. This pattern of staggering lengths is effective in mitigating overcrowding of the cilia, and allows the catheter to pass through the seal 402 without coring the seal, and without excessive resistance. With respect to specific lengths, the actual length of the cilia will depend on the bore size of the Tuohy; however in general, normalizing the lengths to the long cilium 406a, the medium cilium 406b is between 90% and 60% of 406a, and the short cilium is between 90% and 60% of 460b.

The embodiments described herein are examples of a more general invention. A dynamic radial Touhy-Borst seal is provided that comprises two basic components. One is a means for seating, connecting, or mounting the seal within a Tuohy-Borst adapter so that it remains fixed in an operative position while acted upon by fluid pressure and microcatheters. The annular base flange is an example of such a mean for seating the seal. Another basic component is a plurality of pliable occlusive elements that are inwardly directed within the means for mounting. The occlusive elements flex when acted upon by a microcatheter or similar device, allowing it to pass through. But, the occlusive elements are sufficiently rigid to block blood flow through the seal. Furthermore, the seal is dynamic, meaning it forms an effective seal against e.g., a microcatheter, even when the microcatheter is moving relative to the seal. The occlusive elements described herein include various arrangements of rubber flaps, overlapping rubber flaps, and cilia. However, the invention is not limited to such structures. Occlusive elements can take other forms as well provided that they perform the necessary functions of, as noted above, cooperating with a Tuohy-Borst adapter, and dynamically sealing against blood flow at physiological pressures. The person having ordinary skill in the art will readily understand the full scope of the term “occlusive elements” upon reading and understanding the teachings set forth herein.

It will be apparent to those skilled in the art that the above methods and apparatuses may be changed or modified without departing from the general scope of the invention. The invention is intended to include all such modifications and alterations insofar as they come within the scope of the appended claims or the equivalents thereof.

Having thus described the invention, it is now claimed:

I claim:

1. A dynamic radial Tuohy-Borst seal, comprising:
an annular base flange having an annular planar surface oriented radially inward; and
a plurality of pliable cilia radiating inward from the annular planar surface, and arranged in a regular pattern relative to each other on the annular planar surface, wherein the plurality of pliable cilia form an occlusion in a liquid flow path of the annular base flange with a breaking pressure above physiological blood pressure.

2. The dynamic radial Tuohy-Borst seal of claim 1, wherein the plurality of pliable cilia are each characterized by one of a plurality of predetermined lengths from a base of a cilium to a tip of a cilium.

3. The dynamic radial Tuohy-Borst seal of claim 1, wherein plurality of predetermined lengths range from 90% of the length of a longest cilium to 60% of the length of a medium cilium.

4. A dynamic radial Touhy-Borst seal, comprising:
an annular base flange having an annular planar surface oriented radially inward; and
a plurality of inwardly directed pliable occlusive elements together forming an occlusion with a breaking pressure above physiological blood pressure.
